(19) United States
(12) Patent Application Publication
(10) Pub. No.: US 2025/0290744 A1
(43) Pub. Date: Sep. 18, 2025

(54) INTERFERENCE FRINGE PROJECTION OPTICAL SYSTEM, SHAPE MEASUREMENT DEVICE, AND SHAPE MEASUREMENT METHOD

(71) Applicant: OLYMPUS MEDICAL SYSTEMS CORP., Tokyo (JP)

(72) Inventors: Daichi WATANABE, Hachioji-shi (JP); Toshiaki SUZUKI, Hachioji-shi (JP)

(73) Assignee: OLYMPUS MEDICAL SYSTEMS CORP., Tokyo (JP)

(21) Appl. No.: 19/226,406

(22) Filed: Jun. 3, 2025

Related U.S. Application Data
(63) Continuation of application No. PCT/JP2022/044936, filed on Dec. 6, 2022.

(51) Int. Cl. G01B 11/24

(52) U.S. Cl. CPC G01B 11/2441

(57) ABSTRACT

A light distribution correction lens on which a luminous flux emitted from the light emitting unit is incident and which is a single lens having a positive power is arranged at a position at which a distance to the light emitting unit is smaller than a distance to an incidence-side lens group.

A focal length of the incidence-side lens group is denoted by f1 and a focal length of the emission-side lens group is denoted by f2, f1/f2>3 is satisfied, and each of the incidence-side lens group and the emission-side lens group has positive refractive power, and when a distance from an emission-side principal point of the incidence-side lens group to an incidence-side principal point of the emission-side lens group is denoted by xd, xd<f1+f2 is satisfied.

### CROSS REFERENCE TO RELATED APPLICATIONS

This application is a continuation application based on a PCT Patent Application No. PCT/JP2022/044936, filed Dec. 6, 2022, the entire content of which are hereby incorporated by reference.

### BACKGROUND OF THE INVENTION

### Field of the Invention

The present disclosure relates to an interference fringe projection optical system, a shape measurement device, and a shape measurement method.

### Description of the Related Art

As a device that projects an interference fringe, for example, a shape measurement device including an interference fringe projection optical system, an imaging unit, and a calculation unit has been proposed (for example, refer to Patent Japanese Patent No. 6586459).

The shape measurement device projects an interference fringe onto an object surface of a measurement target by the interference fringe projection optical system, images the projected interference fringe by the imaging unit, and analyzes the imaged interference fringe by the calculation unit to measure a three-dimensional shape of the object surface.

### SUMMARY

In a magnification optical system that projects two divergent luminous fluxes whose principal rays emitted from a light source are parallel, the two luminous fluxes have coherence. When a focal length of an incidence-side lens group is denoted by f1 and a focal length of an emission-side lens group is denoted by f2 and a distance from an emission-side principal point of the incidence-side lens group to an incidence-side principal point of the emission-side lens group is xd, in a case where xd/(f1+f2)=1 is not satisfied, the principal rays of the two luminous fluxes emitted from the optical system cannot be parallel (are not image side telecentric), and thus the size of an entrance pupil is reduced. Therefore, a difference in wavefront aberration of the two luminous fluxes on the pupil plane increases, and the interference fringe is distorted. In particular, in a case where the magnification optical system is to be reduced in size, it is necessary to satisfy xd/(f1+f2)<1. Therefore, the telecentricity is not maintained, and the interference fringe is distorted.

Japanese Patent No. 6586459 discloses a range of a surface distance and a focal length between lens groups in which a decrease in contrast caused by the fact that the principal rays cannot be parallel to each other as described above is less likely to occur. However, a configuration for suppressing distortion of the interference fringe caused by the non-parallelism of the principal rays is not disclosed.

The present disclosure has been made in consideration of such circumstances, and an object of the present disclosure is to provide an interference fringe projection optical system, a shape measurement device, and a shape measurement method that can suppress occurrence of distortion of interference fringes even in a case where the optical system is reduced in size.

An interference fringe projection optical system according to a first aspect of the present disclosure includes a light source configured to generate an interference fringe, and a magnification optical system configured to magnify the interference fringe and project the interference fringe onto an object surface, in which the light source includes two light emitting units that are positioned side by side on an incident side of the magnification optical system.

The magnification optical system includes a light distribution correction lens on which a luminous flux emitted from the light emitting unit is incident and which is a single lens having a positive power, an incidence-side lens group which is arranged on a side on which a luminous flux emitted from the light distribution correction lens is incident, and an emission-side lens group which is arranged on a side on which the interference fringe is projected toward the object surface by emitting the luminous flux.

The light distribution correction lens is arranged at a position at which a distance from the light distribution correction lens to the light emitting unit is smaller than a distance from the light distribution correction lens to the incidence-side lens group.

When a focal length of the incidence-side lens group is denoted by f1, and a focal length of the emission-side lens group is denoted by f2, f1/f2>3 is satisfied.

Each of the incidence-side lens group and the emission-side lens group has positive refractive power, and when a distance from an emission-side principal point of the incidence-side lens group to an incidence-side principal point of the emission-side lens group is denoted by xd, xd<f1+f2 is satisfied.

A shape measurement device according to a second aspect of the present disclosure includes: an interference fringe projection optical system including a light source which generates an interference fringe, and a magnification optical system which magnifies the interference fringe and projects the interference fringe onto an object surface, in which the light source includes two light emitting units that are positioned side by side on an incident side of the magnification optical system, the magnification optical system includes a light distribution correction lens on which a luminous flux emitted from the light emitting unit is incident and which is a single lens having a positive power, an incidence-side lens group which is arranged on a side on which a luminous flux emitted from the light distribution correction lens is incident, and an emission-side lens group which is arranged on a side on which the interference fringe is projected toward the object surface by emitting the luminous flux, the light distribution correction lens is arranged at a position at which a distance from the light distribution correction lens to the light emitting unit is smaller than a distance from the light distribution correction lens to the incidence-side lens group, when a focal length of the incidence-side lens group is denoted by f1 and a focal length of the emission-side lens group is denoted by f2, f1/f2>3 is satisfied, and each of the incidence-side lens group and the emission-side lens group has positive refractive power, and when a distance from an emission-side principal point of the incidence-side lens group to an incidence-side principal point of the emission-side lens group is denoted by xd, xd<f1+f2 is satisfied; an imaging unit configured to capture, as an image, the interference fringe projected onto the object surface on an emission side with respect to an image of a light emitting unit; and a calculation unit configured to compute unevenness information of the object surface based on an image signal from the imaging unit.

A shape measurement method according to a third aspect of the present disclosure using an interference fringe projection optical system including a light source which generates an interference fringe, and a magnification optical system which magnifies the interference fringe and projects the interference fringe onto an object surface, in which the light source includes two light emitting units that are positioned side by side on an incident side of the magnification optical system, the magnification optical system including a light distribution correction lens on which a luminous flux emitted from the light emitting unit is incident and which is a single lens having a positive power, an incidence-side lens group which is arranged on a side on which a luminous flux emitted from the light distribution correction lens is incident, and an emission-side lens group which is arranged on a side on which the interference fringe is projected toward the object surface by emitting the luminous flux, the light distribution correction lens is arranged at a position at which a distance from the light distribution correction lens to the light emitting unit is smaller than a distance from the light distribution correction lens to the incidence-side lens group, when a focal length of the incidence-side lens group is denoted by f1 and a focal length of the emission-side lens group is denoted by f2, f1/f2>3 is satisfied, and each of the incidence-side lens group and the emission-side lens group has positive refractive power, and when a distance from an emission-side principal point of the incidence-side lens group to an incidence-side principal point of the emission-side lens group is denoted by xd, xd<f1+f2 is satisfied, the shape measurement method including: capturing, as an image, the interference fringe projected onto the object surface on an emission side with respect to an image of the light emitting unit; and calculating unevenness information of the object surface based on an image signal from the captured image.

According to the above-described aspect, even in a case where an interference fringe projection optical system is reduced in size, occurrence of distortion of an interference fringe can be suppressed.

### BRIEF DESCRIPTION OF THE DRAWINGS

FIG. 1 is a schematic configuration diagram of a main part of a shape measurement device according to a first embodiment of the present disclosure.

FIG. 2 is a diagram showing a light source of an interference fringe projection optical system of FIG. 1.

FIG. 3 is a diagram showing a first modification example of the interference fringe projection optical system of the present disclosure.

FIG. 4 is a diagram showing a second modification example of the interference fringe projection optical system of the present disclosure.

FIG. 5 is a diagram showing a third modification example of the interference fringe projection optical system of the present disclosure.

FIG. 6 is a diagram showing a fourth modification example of the interference fringe projection optical system of the present disclosure.

FIG. 7 is a diagram showing a form of holding a plano-convex lens in the interference fringe projection optical system of FIG. 6.

FIG. 8 is a diagram showing a fifth modification example of the interference fringe projection optical system of the present disclosure.

FIG. 9 is a diagram showing a sixth modification example of the interference fringe projection optical system of the present disclosure.

FIG. 10 is a diagram showing a seventh modification example of the interference fringe projection optical system of the present disclosure.

FIG. 11 is a schematic configuration diagram of an endoscope using a shape measurement device according to a second embodiment of the present disclosure.

FIG. 12 is a schematic configuration diagram of an endoscope system using a shape measurement device according to a third embodiment of the present disclosure.

FIG. 13 is a diagram showing a projection probe used in the endoscope system of FIG. 12.

FIG. 14 is a cross-sectional view of main parts showing a state in which the projection probe of FIG. 13 is inserted into the endoscope.

FIG. 15 is a cross-sectional view of main parts showing a state in which a projection probe is inserted into an endoscope in an endoscope system using a shape measurement device according to a fourth embodiment of the present disclosure.

### DETAILED DESCRIPTION OF THE PREFERRED EMBODIMENTS

### First Embodiment

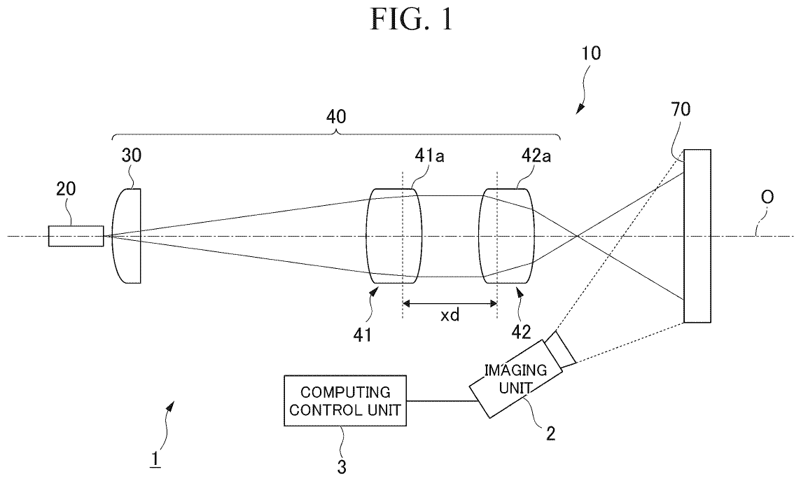

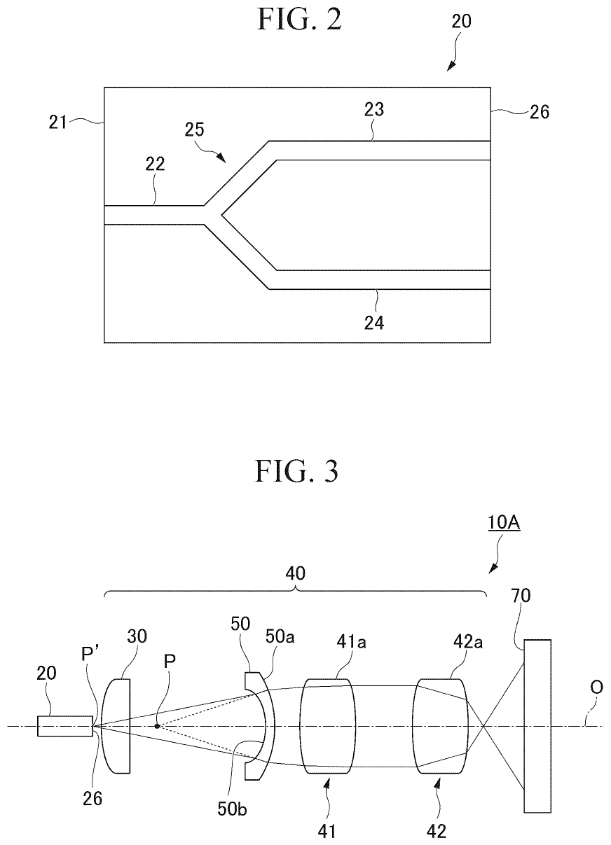

Hereinafter, a configuration of a shape measurement device using an interference fringe projection optical system according to a first embodiment will be described with reference to FIGS. 1 and 2.

FIG. 1 is a schematic configuration diagram of a main part of a shape measurement device according to the first embodiment. The shape measurement device 1 includes an interference fringe projection optical system 10, an imaging unit 2, and a calculation control unit (calculation unit) 3 including a computer or the like.

The interference fringe projection optical system 10 includes a light source 20 and a magnification optical system 40.

In the present embodiment, a planar lightwave circuit (PLC) is used as the light source 20. As shown in FIG. 2, in the PLC, for example, a waveguide structure is formed on a silicon substrate 21 using a quartz-based material. The configuration of the light source 20 is not particularly limited, and for example, an input waveguide 22, a first waveguide 23, a second waveguide 24, and an optical coupler 25 are formed on a silicon substrate 21. The first waveguide 23 and the second waveguide 24 are formed in parallel. The light input into the input waveguide 22 is branched into the first waveguide (light emitting unit) 23 and the second waveguide (light emitting unit) 24 by the optical coupler 25, and the coherent light is emitted from the first waveguide 23 and the second waveguide 24. The light to be input into the input waveguide 22 is, for example, laser light having a single wavelength.

For example, a heater (not shown) is arranged in the vicinity of the first waveguide 23, and a phase difference between two luminous fluxes emitted from the first waveguide 23 and the second waveguide 24 is changed by the heater. The configuration of changing the phase difference between the two luminous fluxes emitted from the first waveguide 23 and the second waveguide 24 is not limited to the heater. For example, a difference in a running path length between the two luminous fluxes to be interfered with each other may be changed by using a movable mirror, and various known configurations can be applied.

An interval between the first waveguide 23 and the second waveguide 24 in an arrangement direction is about 50 μm.

In FIG. 1, the principal rays of the luminous fluxes emitted from the two first waveguides 23 and second waveguide 24 are actually two, but the principal rays of the two luminous fluxes are very close to each other. Therefore, in the following description, only one of the principal rays of the two luminous fluxes is shown.

As shown in FIG. 1, the magnification optical system 40 includes a light distribution correction lens 30, an incidence-side lens group 41, and an emission-side lens group 42. The incidence-side lens group 41 includes a single lens 41a having a positive refractive power. Similarly, the emission-side lens group 42 also includes a single lens 42a having a positive refractive power.

As shown in FIG. 1, two luminous fluxes emitted from the light distribution correction lens 30 are magnified by the magnification optical system 40 and are projected onto the object surface 70. In order to magnify the luminous flux emitted from the light source 20, the magnification optical system 40 makes a focal length f1 of the lens 41a larger than a focal length f2 of the lens 42a. In the present embodiment, a relationship between the focal length f1 and the focal length f2 satisfies the following Expression (1).

$$[ Expression 1 ]$$

$$f 1 / f 2 > 3 \qquad ( 1 )$$

In order to perform the shape measurement over a wide range of the object surface 70, it is necessary to spread the luminous flux over a wide range, and it is preferable that f1/f2 is more than 3 in order to spread the luminous flux. In a case where a beam radius (a radius at which energy is 1/e²) w₀ of a luminous flux emitted from a waveguide end surface of the light source 20 is set to 1.5 μm and a wavelength is set to 520 nm, a divergence angle is obtained from the following Expression (2), and NA=0.110 is obtained from NA=sin θ. In a case where a half angle of view of an imaging system is set to 70 deg (NA=0.940), the magnification of the projection optical system needs to be 8.5 times (0.940/0.110). Therefore, f1/f2 may be more than 8.5. Further, in a case where the illumination is to be performed in a region where the energy is 1/e, it can be seen that f1/f2 may be more than 12 by performing the same calculation using the following Expression (3). Accordingly, the measurement range can be adapted to an angle of view of the imaging system, which is preferable.

$$[ Expression 2 ]$$

$$θ ≃ \frac{λ}{π {w}_{0}} \qquad ( 2 )$$

$$[ Expression 3 ]$$

$$w = \sqrt{2} {w}_{0} \qquad ( 3 )$$

The light distribution correction lens 30 is arranged between the light source 20 and the incidence-side lens group 41 in the magnification optical system 40. A distance between the light distribution correction lens 30 and the light sources (the first waveguide 23 and the second waveguide 24) 20 is shorter than a distance between the light distribution correction lens 30 and the incidence-side lens group 41. That is, the light distribution correction lens 30 is arranged close to the light source 20.

The light distribution correction lens 30 is a single lens on which the luminous flux emitted from the light source 20 is incident and which has a positive power (convex lens). The effect of the light distribution correction lens 30 is that the principal ray of the luminous flux emitted from the light source 20 is focused in front, and the focus thereof coincides with a front focal point of the emission-side lens group 42. As a result, the principal ray of the luminous flux emitted from the light distribution correction lens 30 and passed through the magnification optical system 40 is parallel (telecentric). That is, it is preferable that the lens has a positive power (a convex lens) in order to focus the front side.

Since the light distribution correction lens 30 is a single lens, the total length of the interference fringe projection optical system 10 in the direction of an optical axis O is shortened. Since a single-wavelength laser is used as the light source 20, the influence of chromatic aberration by the light distribution correction lens 30 can be suppressed as much as possible.

In addition, since the interval between the first waveguide 23 and the second waveguide 24 is very small (about 50 μm), the principal rays of the two luminous fluxes emitted from the light source 20 pass through the vicinity of the optical axis O. Therefore, even in a case where the light distribution correction lens 30 is used as a single lens, occurrence of aberration can be suppressed.

The incidence-side lens group 41 is arranged such that the luminous flux emitted from the light distribution correction lens 30 is incident. The incidence-side lens group 41 converts the luminous flux emitted from the light distribution correction lens 30 into substantially collimated light.

The emission-side lens group 42 is arranged such that the luminous flux emitted from the incidence-side lens group 41 is incident. The emission-side lens group 42 condenses the luminous flux emitted from the incidence-side lens group 41. The luminous flux that is once condensed by the emission-side lens group 42 is subsequently diffused to illuminate a wide region.

When an interval between principal point positions of the incidence-side lens group 41 and the emission-side lens group 42, that is, a distance from the emission-side principal point of the incidence-side lens group 41 to the incidence-side principal point of the emission-side lens group 42 is denoted by xd, the following Expression (4) is satisfied.

$$[ Expression 4 ]$$

$$xd < f 1 + f 2 \qquad ( 4 )$$

The incidence-side lens group 41 and the emission-side lens group 42 are arranged to satisfy the above-mentioned Expression (4).

The imaging unit 2 captures, as an image, the interference fringe projected onto the object surface on the emission side with respect to an image of the light emitting unit. The calculation control unit 3 computes unevenness information (three-dimensional shape) of the object surface based on the image signal from the imaging unit 2.

Next, the action of the shape measurement device using the interference fringe projection optical system configured as described above will be described.

The light input into the input waveguide 22 of the light source 20 is branched by the optical coupler 25. The branched light passes through the first waveguide 23 and the second waveguide 24. Since the phase of the luminous flux passing through the first waveguide 23 is controlled by the heater, two luminous fluxes having different phases are emitted from the first waveguide 23 and the second waveguide 24 in a substantially parallel manner.

The luminous flux emitted from the light source 20 is magnified by the incidence-side lens group 41 and the emission-side lens group 42 constituting the magnification optical system 40, and the interference fringe is projected onto the object surface 70 of the measurement target.

The shape measurement device according to the present embodiment projects an interference fringe on the object surface 70 by the interference fringe projection optical system 10, and images the interference fringe on the object surface 70 by the imaging unit 2 while scanning the interference fringe with an interference fringe scanning unit (not shown). The image signal is taken into the calculation control unit 3. Then, the calculation control unit 3 computes the unevenness information of the object surface 70 based on the image signal of the interference fringe to measure the three-dimensional shape.

According to the present embodiment, the light distribution correction lens 30 is a single lens that has a positive power and that is incident with the luminous flux emitted from the first and second waveguides 23 and 24, and is arranged at a position at which a distance to the first and second waveguides 23 and 24 is smaller than a distance to the incidence-side lens group 41. With this configuration, the two luminous fluxes emitted from the light source 20 and having passed through the magnification optical system 40 are substantially parallel to each other. Therefore, the entrance pupil is substantially infinite, and the shift amount of the principal rays of the two luminous fluxes on the pupil plane can be ignored. Therefore, wavefront shapes of the two luminous fluxes can be regarded as substantially the same. Therefore, since the difference in wavefront aberration is almost eliminated and the influence of the wavefront aberration can be suppressed, the occurrence of the distortion of the interference fringe can be suppressed.

In addition, even in a case where the distance xd between the incidence-side lens group 41 and the emission-side lens group 42 is reduced, in other words, even in a case where the incidence-side lens group 41 and the emission-side lens group 42 are brought close to each other, the occurrence of the distortion of the interference fringe can be suppressed. Therefore, it is possible to suppress the distortion of the interference fringe projected onto the object surface 70 while reducing the size of the interference fringe projection optical system 10.

In addition, since the principal ray of the luminous flux emitted from the emission-side lens group 42 is substantially parallel to the optical axis O, the entrance pupil is substantially infinite. As a result, the robustness of the distortion of the interference fringe with respect to the wavefront aberration on the object surface 70 is improved.

Further, even in a case where the object surface 70 is positioned at a position away from the shape measurement device 1, the interval between the principal rays of the two luminous fluxes can be maintained. Therefore, the contrast of the interference fringe can be maintained high even at a distance.

### First Modification Example

Next, a first modification example according to the present disclosure will be described, but the basic configuration is the same as that of the first embodiment. Therefore, the same reference numerals are given to the same configurations, the description thereof will be omitted, and only the different points will be described.

As shown in FIG. 3, in an interference fringe projection optical system 10A of the present modification example, a concave lens 50 is arranged between the light distribution correction lens 30 and the incidence-side lens group 41.

The concave lens 50 is a meniscus lens having a convex surface facing the incidence-side lens group 41 side. With this configuration, a retrofocus optical system is obtained.

The concave lens 50 is arranged to be aplanatic. The concave lens 50 has a curved surface 50a on the incidence-side lens group 41 side and a curved surface 50b on the light distribution correction lens 30 side. The curved surface 50b of the concave lens 50 is an arc centered at a point P.

In addition, the light refracted by the curved surface 50b of the concave lens 50 coincides with a point P′ on the optical axis O, that is, an emission end surface 26 of the light source 20. In this way, by disposing the concave lens 50 at a position at which the aplanatic condition (condition in which spherical aberration and coma aberration do not occur) is satisfied, aberration can be suppressed.

According to the present modification example, since the concave lens (meniscus lens) 50 is arranged between the light distribution correction lens 30 and the incidence-side lens group 41, the retrofocus optical system is formed, and the total length of the interference fringe projection optical system 10A can be shortened.

In addition, the concave lens 50 does not necessarily need to be arranged to be aplanatic. However, by disposing the concave lens 50 to be aplanatic, it is possible to provide the interference fringe projection optical system 10A in which the distortion of the interference fringe is suppressed while the interference fringe projection optical system 10A is reduced in size.

Further, by disposing the concave lens 50 to be aplanatic, wavefront aberration can be suppressed. That is, there is almost no change in aberration between the optical system in which the concave lens 50 is arranged and the optical system in which the concave lens 50 is not arranged.

Therefore, in a case where the total lengths of the optical systems are substantially the same between the optical system in which the concave lens 50 is arranged and the optical system in which the concave lens 50 is not arranged, an image-forming magnification can be changed. Therefore, it is possible to realize an optical system having a different illumination angle of view in a state where the optical systems other than the concave lens 50 are common. For example, in general, in an endoscope, an angle of view (140 degrees) of an upper endoscope is smaller than an angle of view (170 degrees) of a lower endoscope. Therefore, in the upper endoscope, the concave lens 50 is not arranged, and in the lower endoscope, the concave lens 50 is arranged, so that the image-forming magnification can be changed, and thus the optical system other than the concave lens 50 can be shared. As a result, it is possible to reduce the manufacturing cost.

### Second Modification Example

Next, a second modification example according to the present disclosure will be described, but the basic configuration is the same as that of the first embodiment. Therefore, the same reference numerals are given to the same configurations, the description thereof will be omitted, and only the different points will be described.

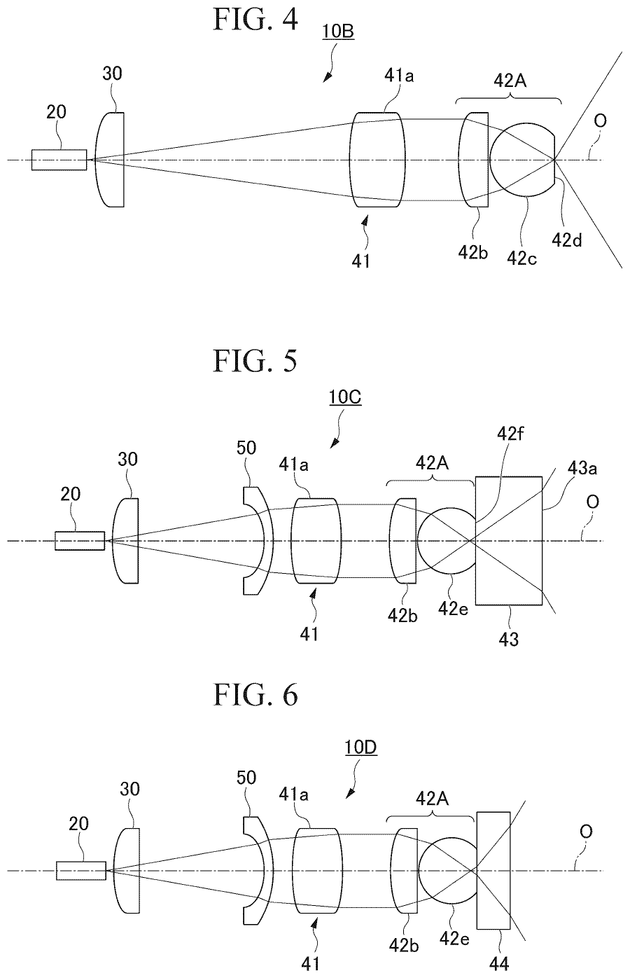

As shown in FIG. 4, in an interference fringe projection optical system 10B of the present modification example, the configuration of an emission-side lens group 42A is different from that of the first embodiment.

The emission-side lens group 42A includes a lens 42b and a plano-convex lens 42c.

The lens 42b is arranged such that the luminous flux emitted from the incidence-side lens group 41 is incident thereon, and is a plano-convex lens having a convex surface facing the incidence-side lens group 41 side.

The plano-convex lens 42c has a shape in which a part of a ball lens is cut. The plano-convex lens 42c is arranged such that the luminous flux emitted from the lens 42b is incident thereon, and is a lens having a convex surface facing the lens 42b.

The plano-convex lens 42c is arranged to be aplanatic. The plano-convex lens 42c is formed such that the incident luminous flux is condensed on an emission end surface (flat surface) 42d. That is, a focal point of the plano-convex lens 42c is located at the emission end surface 42d.

According to the present modification example, since a ball lens that can be manufactured at low cost can be used as the plano-convex lens 42c, the manufacturing cost can be reduced. Further, by disposing the plano-convex lens 42c to be aplanatic, the interference fringe projection optical system 10B in which distortion of the interference fringe is suppressed can be provided.

As in the first modification example, the concave lens 50 may be arranged between the light distribution correction lens 30 and the incidence-side lens group 41.

### Third Modification Example

Next, a third modification example according to the embodiment of the present disclosure will be described, but the basic configuration is the same as that of the second modification example. Therefore, the same reference numerals are given to the same configurations, the description thereof will be omitted, and only the different points will be described.

As shown in FIG. 5, in an interference fringe projection optical system 10C of the present modification example, a concave lens (meniscus lens) 50 is arranged between the light distribution correction lens 30 and the incidence-side lens group 41.

A plano-convex lens 42e has a shape in which a part of a ball lens is cut. The plano-convex lens 42e is formed by cutting a ball lens such that the focal position does not coincide with an emission end surface 42f. In the present modification example, a curvature radius, a refractive index, and a thickness of the plano-convex lens 42e are defined such that the focal point is positioned on the optical axis O inside the plano-convex lens 42e.

A flat plate glass 43 is provided in contact with the emission end surface 42f of the plano-convex lens 42e. The flat plate glass 43 is arranged such that the luminous flux emitted from the emission-side lens group 42A is incident. In the present modification example, the plano-convex lens 42e and the flat plate glass 43 are adhered to each other by an adhesive.

The refractive index of the plano-convex lens 42e and the refractive index of the flat plate glass 43 are the same.

For example, in a case where the flat plate glass 43 is not used for the emission end surface 42f of the plano-convex lens 42e, since the energy is concentrated on the emission end surface 42f, when a liquid adheres to the emission end surface 42f, the liquid is baked on the emission end surface 42f. In order to prevent this, in the present modification example, the flat plate glass 43 is arranged on the emission end surface 42f. Since an outer diameter of the luminous flux emitted from the flat plate glass 43 can be increased by the flat plate glass 43, the energy density at the emission end surface 43a of the flat plate glass 43 can be reduced.

According to the present modification example, the plano-convex lens 42e is formed such that the focal position of the plano-convex lens 42e does not coincide with the emission end surface 42f and the focal point is positioned on the optical axis O inside the plano-convex lens 42e. That is, since the luminous flux is not focused on the position of the adhesive between the plano-convex lens 42e and the flat plate glass 43, the photodegradation of the adhesive can be prevented.

In addition, although spherical aberration occurs by disposing the flat plate glass 43, the entrance pupil can be enlarged since the light distribution correction lens 30 is arranged. As a result, the influence of the distortion of the interference fringe on the aberration caused by the flat plate glass 43 can be suppressed.

Further, since the concave lens 50 is arranged between the light distribution correction lens 30 and the incidence-side lens group 41, the total length of the interference fringe projection optical system 10C in the optical axis direction can be shorter than that in the second modification example.

Due to the effect of improving robustness by the light distribution correction lens 30, the influence of the spherical aberration generated in the flat plate glass 43 can be ignored.

### Fourth Modification Example

Next, a fourth modification example according to the present disclosure will be described, but the basic configuration is the same as that of the third modification example. Therefore, the same reference numerals are given to the same configurations, the description thereof will be omitted, and only the different points will be described.

As shown in FIG. 6, in an interference fringe projection optical system 10D of the present modification example, the refractive indices of the plano-convex lens 42e and the flat plate glass 44 are different from each other.

In a case where the refractive index of the plano-convex lens 42e is denoted by n1 and the refractive index of the flat plate glass 44 is denoted by n2, n1>n2 is satisfied.

With this configuration, the luminous flux emitted from the plano-convex lens 42e is refracted by the flat plate glass 44. As a result, even in a case where the thickness of the flat plate glass 44 in the optical axis O direction is reduced, the divergence angle of the light emitted from the flat plate glass 44 can be maintained.

In addition, the length dimension of the flat plate glass 44 in the optical axis O direction can be reduced as compared with the interference fringe projection optical system 10C of the third modification example shown in FIG. 5. Accordingly, the total length of the interference fringe projection optical system 10D in the optical axis O direction can be further reduced, and thus further reduction in size can be achieved.

Next, a form of holding the plano-convex lens 42e in the interference fringe projection optical system 10D will be described.

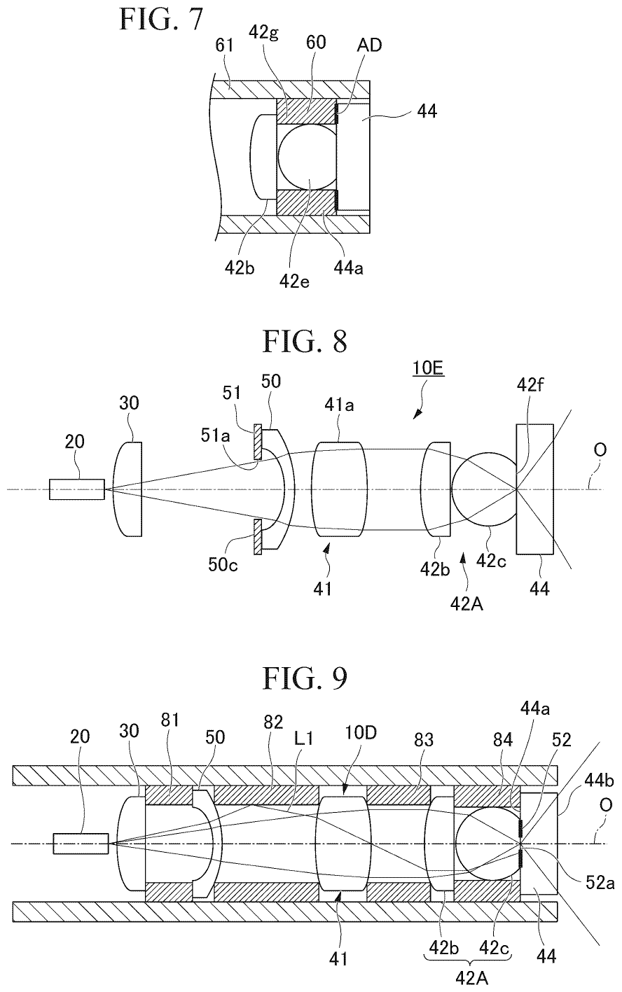

As shown in FIG. 7, the outer diameter of the flat plate glass 44 is larger than the outer diameter of the plano-convex lens 42e. The flat plate glass 44 and the plano-convex lens 42e are bonded to each other by an adhesive AD. The flat plate glass 44 and the plano-convex lens 42e may be bonded to each other not only by an adhesive but also, for example, by optical contact.

A spacing ring 60 is provided between the lens 42b and the flat plate glass 44. The spacing ring 60 is formed in a tubular shape, and the plano-convex lens 42e is arranged therein. The first end of the spacing ring 60 is bonded to the emission end surface 42g of the lens 42b, and the second end of the spacing ring 60 is bonded to an incident end surface 44a of the flat plate glass 44.

A mirror frame 61 is formed in a tubular shape, and the spacing ring 60 is held inside the mirror frame 61. In the present modification example, an example in which the mirror frame 61 and the spacing ring 60 are separate bodies has been described, but the mirror frame 61 and the spacing ring 60 may be integrally configured.

In general, it is difficult to hold a plano-convex lens (ball lens), but in the present modification example, since the flat plate glass 44 to which the plano-convex lens 42e is bonded is held by the spacing ring 60, the mounting work is facilitated. Further, in a case where the axial deviation of the plano-convex lens 42e occurs, the luminous flux is emitted obliquely. However, since the axial deviation of the plano-convex lens 42e can be suppressed, the deviation of the projection position of the interference fringe projected onto the object surface 70 can be reduced.

### Fifth Modification Example

Next, a fifth modification example according to the present disclosure will be described, but the basic configuration is the same as that of the fourth modification example. Therefore, the same reference numerals are given to the same configurations, the description thereof will be omitted, and only the different points will be described.

An interference fringe projection optical system 10E of the present modification example includes a light shielding plate 51.

As shown in FIG. 8, the light shielding plate 51 has an opening portion 51a formed in the center. The light shielding plate 51 is arranged between the light distribution correction lens 30 and the concave lens 50. In the present modification example, the light shielding plate 51 is bonded to an incidence-side end surface 50c of the concave lens 50.

An end surface 50c of the concave lens 50 outside an effective diameter of the incident surface is a flat surface, and the light shielding plate 51 abuts on the flat surface. With this configuration, the light shielding plate 51 can be easily mounted on the concave lens 50.

In addition, the plano-convex lens 42c in which the luminous flux emitted from the light source 20 is condensed on the emission end surface 42f of the plano-convex lens 42c is used. As shown in FIG. 6, the plano-convex lens 42e that does not condense the luminous flux emitted from the light source 20 to the emission end surface 42f of the plano-convex lens 42c may be used.

According to the present modification example, in the light emitted from the light distribution correction lens 30, unnecessary light is shielded by the light shielding plate 51, and necessary light passes through the opening portion 51a of the light shielding plate 51 and is incident on the concave lens 50. As a result, by shielding the luminous flux outside the desired luminous flux, the occurrence of stray light can be prevented, and the occurrence of unintended illumination unevenness (collapse of the fringe pattern) can be suppressed. Further, since the light shielding plate 51 is arranged between the light distribution correction lens 30 and the concave lens 50, it is possible to suppress the occurrence of stray light before a large amount of luminous flux passes through the plurality of optical elements. As a result, the cause of the occurrence of unnecessary (unintended) stray light can be removed at an early stage.

In addition, as shown in the above-described fourth modification example, in a case where the interference fringe projection optical system is held by using the mirror frame and the spacing ring, the mirror frame and the spacing ring may be subjected to an antireflection treatment. Examples of the antireflection treatment include black coating, black plating, and black dyeing. In this way, by performing the antireflection treatment on the mirror frame and the spacing ring, the unintended light reflected by the mirror frame and the spacing ring is absorbed, and thus the occurrence of stray light can be suppressed.

The light shielding plate 51 is arranged between the light distribution correction lens 30 and the concave lens 50, but the light shielding plate 51 may be arranged between the light distribution correction lens 30 and the incidence-side lens group 41.

### Sixth Modification Example

Next, a sixth modification example according to the embodiment of the present disclosure will be described, but the basic configuration is the same as that of the fourth modification example. Therefore, the same reference numerals are given to the same configurations, the description thereof will be omitted, and only the different points will be described.

The present modification example includes a mirror frame and a spacing ring that hold the interference fringe projection optical system 10D of the fourth modification example.

As shown in FIG. 9, a tubular spacing ring 81 is provided between the light distribution correction lens 30 and the concave lens 50, a tubular spacing ring 82 is provided between the concave lens 50 and the incidence-side lens group 41, a tubular spacing ring 83 is provided between the incidence-side lens group 41 and the lens 42b, and a tubular spacing ring 84 is provided between the lens 42b and the flat plate glass 44.

The optical elements (the light distribution correction lens 30, the concave lens 50, the incidence-side lens group 41, the lens 42b, and the plano-convex lens 42c) are held by the spacing rings 81 to 84 while maintaining a predetermined interval in the optical axis O direction of each optical element. Examples of a material of the spacing rings 81 to 84 include glass and ceramics.

The mirror frame 85 is formed in a tubular shape, and the spacing rings 81 to 84 are held inside the mirror frame 85. In the present modification example, although an example in which the mirror frame 85 and the spacing rings 81 to 84 are separate bodies has been described, the mirror frame 85 and the spacing rings 81 to 84 may be integrally configured.

Here, in a case where the material of the spacing ring is formed of a metal, light L1 that reaches the spacing ring is scattered light. However, in the present modification example, since the materials of the spacing rings 81 to 84 are formed of glass or ceramics, the light that reaches the spacing rings 81 to 84 is reflected light instead of scattered light. As a result, the ray can be easily controlled by design. Furthermore, only the intended luminous flux can be preferentially passed through the focusing point.

In addition, a light shielding mask 52 is provided between the plano-convex lens 42c and the flat plate glass 44. An opening portion 52a is formed at the center of the light shielding mask 52. The luminous flux emitted from the light source 20 passes through the opening portion 52a.

The light shielding mask 52 is arranged such that the focusing point of the luminous flux is positioned in the opening portion 52a. That is, it is preferable that the light shielding mask 52 is arranged in the vicinity of the focusing point. With this configuration, since the light L1 reflected by the spacing ring 82 is shielded by the light shielding mask 52, the occurrence of illumination unevenness (collapse of the fringe pattern) can be suppressed.

A method of forming the light shielding mask 52 is not particularly limited, and a pinhole mask may be formed on the emission end surface of the plano-convex lens 42c by Cr vapor deposition or the like. Alternatively, a pinhole mask may be formed on the incident end surface 44a of the flat plate glass 44 on the plano-convex lens 42c side by Cr vapor deposition or the like.

As shown in FIG. 6, the plano-convex lens 42e that does not condense the luminous flux emitted from the light source 20 to the emission end surface 42f of the plano-convex lens 42c may be used.

According to the present modification example, the light emitted from the light source 20 and reflected by, for example, the spacing ring 82 passes through the incidence-side lens group 41 and the emission-side lens group 42A and is shielded by the light shielding mask 52. Accordingly, unnecessary light (light other than the intended luminous flux) can be removed, and thus the distortion of the interference fringe can be further suppressed.

### Seventh Modification Example

Next, a seventh modification example according to the embodiment of the present disclosure will be described, but the basic configuration is the same as that of the fifth modification example. Therefore, the same reference numerals are given to the same configurations, the description thereof will be omitted, and only the different points will be described.

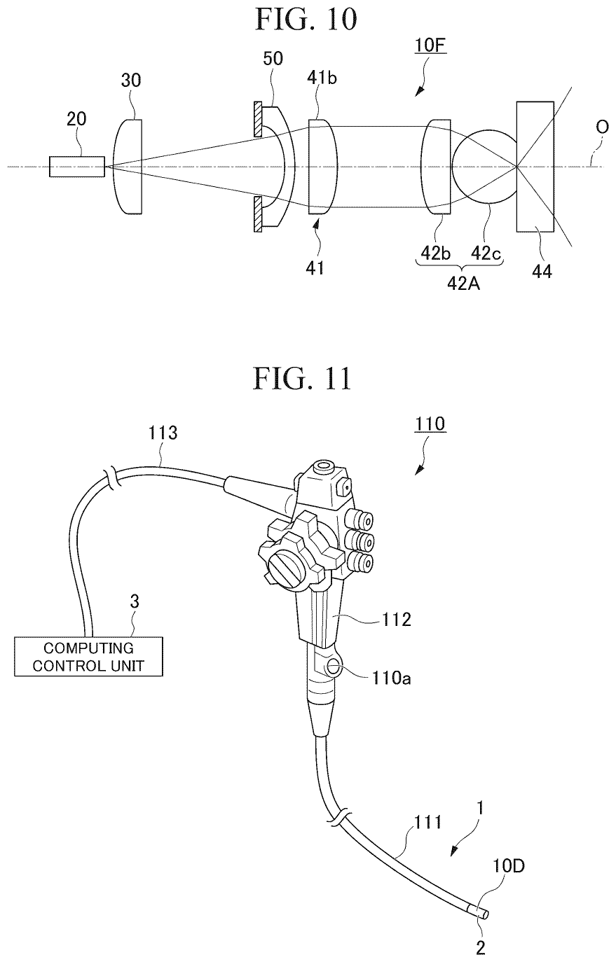

As shown in FIG. 10, in the interference fringe projection optical system 10F of the present modification example, the light distribution correction lens 30 and the lens 41b of the incidence-side lens group 41 are lenses having the same shape and material. The shape and material of the light distribution correction lens 30 and the lens 42b of the emission-side lens group 42A may be the same.

According to the present modification example, since the light distribution correction lens 30 and the lens 41b of the incidence-side lens group 41 are shared, the types of molds and jigs for forming the light distribution correction lens 30 and the lens 41b can be reduced, and batch processing can be performed. Therefore, it is possible to reduce the cost.

### Second Embodiment

Next, a second embodiment according to the present disclosure will be described. In the second embodiment, an endoscope including the shape measurement device 1 having the interference fringe projection optical system 10D of a sixth modification example shown in FIG. 9 will be described. Therefore, the same reference numerals are given to the same configurations, the description thereof will be omitted, and only the different points will be described.

An endoscope 110 is, for example, a device that is used to observe and treat an inside of a body of a patient lying on an operating table. As shown in FIG. 11, the endoscope 110 includes an elongated insertion portion 111 that is to be inserted into the body of the patient, an operation portion 112 that is connected to a base end of the insertion portion 111, and a universal cord 113 that extends from the operation portion 112.

A treatment tool insertion opening 110a is formed on a base end side of the insertion portion 111. The treatment tool insertion opening 110a is connected to a base end portion of a treatment tool insertion channel (not shown) in the insertion portion 111.

The treatment tool insertion opening 110a is an insertion opening through which a treatment tool (not shown) for an endoscope is to be inserted into the treatment tool insertion channel.

The operation portion 112 receives an operation for the endoscope 110. The universal cord 113 connects the endoscope 110 and the calculation control unit 3.

The interference fringe projection optical system 10D and the imaging unit 2 of the sixth modification example are provided at a distal end of the insertion portion 111 of the endoscope 110. The emission end surface 44b of the flat plate glass 44 of the interference fringe projection optical system 10D is positioned at the distal end opening of the insertion portion 111. With this configuration, the interference fringe projected from the interference fringe projection optical system 10D is projected from the emission end surface 44b of the flat plate glass 44 arranged at the distal end of the insertion portion 111 toward the measurement target. The interference fringe is imaged by the imaging unit 2.

The imaging unit 2 captures, as an image, the interference fringe projected onto an object surface on the emission side with respect to the images of the first waveguide 23 and the second waveguide 24. The position of the imaging unit 2 may be provided on the outside of the endoscope 110 instead of the distal end of the insertion portion 111.

The calculation control unit 3 computes unevenness information (three-dimensional shape) of the object surface based on the image signal from the imaging unit 2.

Next, the operation of the endoscope 110 configured as described above will be described.

In a case of measuring the uneven shape inside the body, light is emitted from the interference fringe projection optical system 10D, and the interference fringe is projected onto the uneven shape from the distal end of the insertion portion 111 of the endoscope 110. The imaging unit 2 images the interference fringe, and the image signal is taken into the calculation control unit 3.

Then, the calculation control unit 3 computes unevenness information of the surface of the measurement target based on the image signal of the interference fringe to measure the three-dimensional shape.

According to the present embodiment, since the interference fringe projection optical system 10D that suppresses the distortion of the interference fringe even in a case of being reduced in size is used, it is possible to measure the uneven shape of the measurement target with high accuracy without increasing the dimension of the outer diameter of the endoscope.

### Third Embodiment

Next, a third embodiment according to the present disclosure will be described. In the third embodiment, an endoscope system including a shape measurement device having the interference fringe projection optical system 10E of the fifth modification example will be described. Therefore, the same reference numerals are given to the same configurations, the description thereof will be omitted, and only the different points will be described.

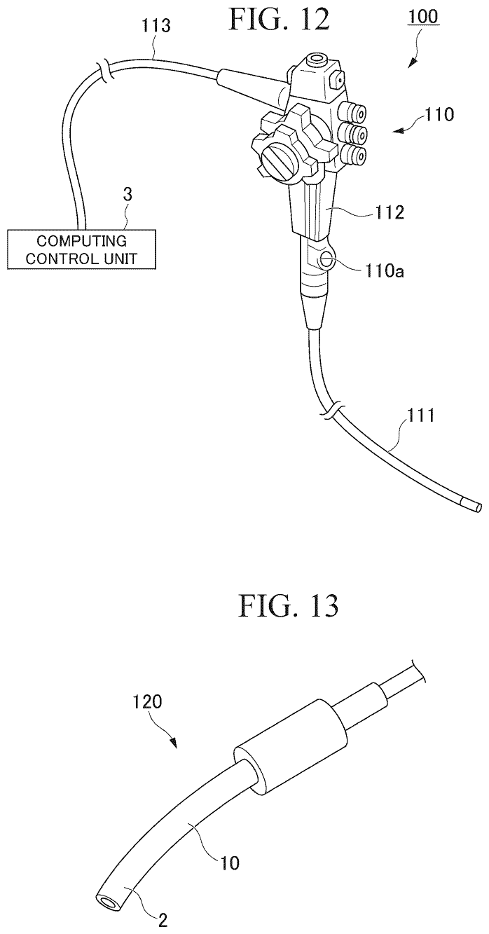

As shown in FIGS. 12 and 13, an endoscope system 100 includes an endoscope 110, a projection probe 120, and a calculation control unit 3. The projection probe 120 includes the interference fringe projection optical system 10E according to the fifth modification example.

As shown in FIG. 12, the endoscope 110 includes an elongated insertion portion 111, an operation portion 112, and a universal cord 113, as in the second embodiment.

A treatment tool insertion opening 110a is an insertion opening for inserting the projection probe 120 of FIG. 13 into the treatment tool insertion channel, in addition to a treatment tool (not shown) for an endoscope.

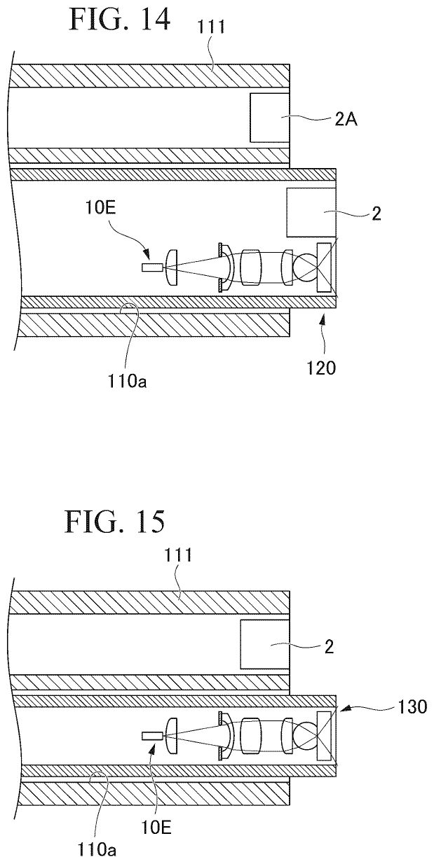

The projection probe 120 projects an interference fringe toward a measurement target. As shown in FIG. 14, the projection probe 120 is provided with the interference fringe projection optical system 10E and the imaging unit 2 of the fifth modification example. With this configuration, the interference fringe projected from the projection probe 120 is imaged by the imaging unit 2.

In addition, as shown in FIG. 14, an endoscope imaging unit 2A is provided at a distal end of the insertion portion 11 of the endoscope 110.

The imaging unit 2 captures, as an image, the interference fringe projected onto an object surface on the emission side with respect to the images of the first waveguide 23 and the second waveguide 24.

The calculation control unit 3 computes unevenness information (three-dimensional shape) of the object surface based on the image signal from the imaging unit 2.

Next, the operation of the endoscope system 100 configured as described above will be described.

In a case of measuring the uneven shape inside the body, as shown in FIG. 14, the projection probe 120 is inserted from the treatment tool insertion opening 110a and pushed to the distal end of the insertion portion 111. After the projection probe 120 protrudes from the distal end of the insertion portion 111, the interference fringe is projected toward the measurement target. The imaging unit 2 images the interference fringe, and the image signal is taken into the calculation control unit 3. Then, the calculation control unit 3 computes unevenness information of the surface of the measurement target based on the image signal of the interference fringe to measure the three-dimensional shape.

According to the present embodiment, since the projection probe 120 in which the distortion of the interference fringe is suppressed even in a case of being reduced in size is used, it is possible to measure the uneven shape of the measurement target with high accuracy without increasing the outer diameter of the endoscope.

### Fourth Embodiment

Next, a fourth embodiment according to the present disclosure will be described. In the fourth embodiment, an endoscope system including the shape measurement device having the interference fringe projection optical system 10E of the fifth modification example will be described. Therefore, the same reference numerals are given to the same configurations, the description thereof will be omitted, and only the different points will be described.

As shown in FIG. 15, in the fourth embodiment, a projection probe 130 is provided with the interference fringe projection optical system 10E of the fifth modification example. An imaging unit 2 is provided at a distal end of an insertion portion 111 of an endoscope 110. With this configuration, the interference fringe projected from a projection probe 130 is imaged by an imaging unit 2. The imaging unit 2 also serves as the endoscope imaging unit 2A of the third embodiment.

In other configurations, the configuration is the same as that of the third embodiment.

According to the present embodiment, since the projection probe 130 does not include the imaging unit 2, the outer diameter of the projection probe 130 can be made smaller than the outer diameter of the projection probe including the imaging unit.

The imaging unit 2 also serves as the endoscope imaging unit 2A of the third embodiment. However, the endoscopic imaging unit 2A may be provided in the endoscope 110 separately from the imaging unit 2.

A technical scope of the present disclosure is not limited to the above-described embodiments, and various modifications can be made without departing from a spirit of the present disclosure.

For example, in the second embodiment, the endoscope including the shape measurement device having the interference fringe projection optical system 10D of the sixth modification example has been described. However, the endoscope may include the shape measurement device using the interference fringe projection optical systems of the first embodiment, the first modification example to the fifth modification example, and the seventh modification example.

In the third and fourth embodiments, the endoscope system including the shape measurement device of the fifth modification example has been described. However, the endoscope system may include the shape measurement devices of the first modification example to the fourth modification example, the sixth modification example, and the seventh modification example using the interference fringe projection optical systems.

In addition, it is possible to replace the components in the above-described embodiments and modification examples with well-known components without departing from the gist of the present disclosure, and the above-described embodiments and modification examples may be appropriately combined.

### INDUSTRIAL APPLICABILITY

According to the interference fringe projection optical system, the shape measurement device, and the shape measurement method of the present disclosure, even in a case where the interference fringe projection optical system is reduced in size, the occurrence of distortion of the interference fringe can be suppressed.

While preferred embodiments of the disclosure have been described and illustrated above, it should be understood that these are exemplary of the disclosure and are not to be considered as limiting. Additions, omissions, substitutions, and other modifications can be made without departing from the scope of the disclosure. Accordingly, the disclosure is not to be considered as being limited by the foregoing description and is only limited by the scope of the appended claims.

What is claimed is:

1. An interference fringe projection optical system comprising:
a light source configured to generate an interference fringe; and
a magnification optical system configured to magnify the interference fringe and project the interference fringe onto an object surface,
wherein the light source includes two light emitting units that are positioned side by side on an incident side of the magnification optical system,
the magnification optical system includes
a light distribution correction lens on which a luminous flux emitted from the light emitting unit is incident and which is a single lens having a positive power,
an incidence-side lens group which is arranged on a side on which a luminous flux emitted from the light distribution correction lens is incident, and
an emission-side lens group which is arranged on a side on which the interference fringe is projected toward the object surface by emitting the luminous flux,
the light distribution correction lens is arranged at a position at which a distance from the light distribution correction lens to the light emitting unit is smaller than a distance from the light distribution correction lens to the incidence-side lens group,
when a focal length of the incidence-side lens group is denoted by f1 and a focal length of the emission-side lens group is denoted by f2, f1/f2>3 is satisfied, and
each of the incidence-side lens group and the emission-side lens group has positive refractive power, and when a distance from an emission-side principal point of the incidence-side lens group to an incidence-side principal point of the emission-side lens group is denoted by xd, xd<f1+f2 is satisfied.

2. The interference fringe projection optical system according to claim 1,
wherein a concave lens is arranged between the light distribution correction lens and the incidence-side lens group.

3. The interference fringe projection optical system according to claim 2,
wherein the concave lens is a meniscus lens and is arranged to be aplanatic.

4. The interference fringe projection optical system according to claim 1,
wherein a lens that is arranged closest to an emission side among lenses constituting the emission-side lens group is arranged to be aplanatic.

5. The interference fringe projection optical system according to claim 1,
wherein when the focal length of the incidence-side lens group is denoted by f1 and the focal length of the emission-side lens group is denoted by f2, f1/f2>8 is satisfied.

6. The interference fringe projection optical system according to claim 5,
wherein when the focal length of the incidence-side lens group is denoted by f1 and the focal length of the emission-side lens group is denoted by f2, f1/f2>12 is satisfied.

7. The interference fringe projection optical system according to claim 1,
wherein a flat plate glass is bonded to an emission end surface of the emission-side lens group.

8. The interference fringe projection optical system according to claim 7,
wherein a refractive index of the emission-side lens group is greater than a refractive index of the flat plate glass.

9. The interference fringe projection optical system according to claim 1,
wherein a light shielding plate having an opening portion is arranged between the light distribution correction lens and the incidence-side lens group, and
the luminous flux emitted from the light distribution correction lens passes through the opening portion.

10. The interference fringe projection optical system according to claim 1,
wherein a light shielding mask having an opening portion is arranged in a vicinity of a condensing position of a luminous flux emitted from the light source on an emission side of the emission-side lens group, and
the luminous flux emitted from the light source passes through the opening portion.

11. The interference fringe projection optical system according to claim 1,
wherein a material and a shape of the light distribution correction lens are the same as a material and a shape of lenses constituting the incidence-side lens group.

12. A shape measurement device comprising:
an interference fringe projection optical system including a light source which generates an interference fringe, and a magnification optical system which magnifies the interference fringe and projects the interference fringe onto an object surface,
in which the light source includes two light emitting units that are positioned side by side on an incident side of the magnification optical system,
the magnification optical system includes
a light distribution correction lens on which a luminous flux emitted from the light emitting unit is incident and which is a single lens having a positive power,
an incidence-side lens group which is arranged on a side on which a luminous flux emitted from the light distribution correction lens is incident, and
an emission-side lens group which is arranged on a side on which the interference fringe is projected toward the object surface by emitting the luminous flux,
the light distribution correction lens is arranged at a position at which a distance from the light distribution correction lens to the light emitting unit is smaller than a distance from the light distribution correction lens to the incidence-side lens group,
when a focal length of the incidence-side lens group is denoted by f1 and a focal length of the emission-side lens group is denoted by f2, f1/f2>3 is satisfied, and
each of the incidence-side lens group and the emission-side lens group has positive refractive power, and when a distance from an emission-side principal point of the incidence-side lens group to an incidence-side principal point of the emission-side lens group is denoted by xd, xd<f1+f2 is satisfied;
an imaging unit configured to capture, as an image, the interference fringe projected onto the object surface on an emission side with respect to an image of a light emitting unit; and
a calculation unit configured to compute unevenness information of the object surface based on an image signal from the imaging unit.

13. A shape measurement method using an interference fringe projection optical system including a light source which generates an interference fringe, and a magnification optical system which magnifies the interference fringe and projects the interference fringe onto an object surface,
in which the light source includes two light emitting units that are positioned side by side on an incident side of the magnification optical system,
the magnification optical system including
a light distribution correction lens on which a luminous flux emitted from the light emitting unit is incident and which is a single lens having a positive power,
an incidence-side lens group which is arranged on a side on which a luminous flux emitted from the light distribution correction lens is incident, and
an emission-side lens group which is arranged on a side on which the interference fringe is projected toward the object surface by emitting the luminous flux,
the light distribution correction lens is arranged at a position at which a distance from the light distribution correction lens to the light emitting unit is smaller than a distance from the light distribution correction lens to the incidence-side lens group,
when a focal length of the incidence-side lens group is denoted by f1 and a focal length of the emission-side lens group is denoted by f2, f1/f2>3 is satisfied, and
each of the incidence-side lens group and the emission-side lens group has positive refractive power, and when a distance from an emission-side principal point of the incidence-side lens group to an incidence-side principal point of the emission-side lens group is denoted by xd, xd<f1+f2 is satisfied, the shape measurement method comprising:
capturing, as an image, the interference fringe projected onto the object surface on an emission side with respect to an image of the light emitting unit; and
calculating unevenness information of the object surface based on an image signal from the captured image.
